(19) United States
(12) Patent Application Publication
(10) Pub. No.: US 2011/0052540 A1
(43) Pub. Date: Mar. 3, 2011

(54) Non-Aggregating Virus Formulation

(76) Inventors: Robert Shaw, Kuopio (FI); Minna Nokelainen, Kuopio (FI); Tuomas Mantyla, Kuopio (FI); Seppo Yla-Herttuala, Kuopio (FI)

(21) Appl. No.: 12/671,950

(22) PCT Filed: Aug. 11, 2008
(86) PCT No.: PCT/GB08/50693

§ 371 Date: Jul. 19, 2010

(30) Foreign Application Priority Data
Aug. 11, 2007 (GB) 0715723.3

(51) Int. Cl. A61K 48/00; C12Q 1/70; A61P 35/00

(52) U.S. Cl. 424/93.6; 435/5

(57) ABSTRACT

The present invention is a composition comprising a virus, a polyol and a zwitteronic compound. The present invention is also an assay for viral aggregation, which comprises analysing the size of the viral particles in a sample, wherein the particles are in admixture with a polyol, and determining from the size whether the sample contains substantially only acceptable, non-aggregated particles.

### FIELD OF THE INVENTION

This invention relates to a virus formulation in which aggregation is minimised.

### BACKGROUND OF THE INVENTION

Viruses can be used to deliver genes, e.g. in gene therapy. Such viruses include retroviruses, adenovirus, adeno-associated virus and herpes simplex virus. For example, an adenovirus that delivers functional thymidine kinase, for use in therapy relating to the treatment of brain tumours and the prevention of their recurrence, is disclosed in WO00/28059.

It will be appreciated that, for therapeutic use, various regulatory requirements must be met. Stringent controls are required on the production of viruses for gene therapy, and the stability of and potency of viral formulations are critical considerations.

WO00/28059 and U.S. Pat. No. 6,544,769 disclose glycerol as a stabiliser of virus formulations. U.S. Pat. No. 7,235,391 discloses glycerol as an additive to viral formulations, for long-term stability at or above refrigeration temperatures. Various other additives are disclosed, including a variety of bulking agents, cryoprotectants, lyoprotectants, buffers etc.

U.S. Pat. No. 6,544,769 discloses a composition comprising virus together with surcrose, glycerol, magnesium chloride and polysorbate 80. The presence of such a surfactant can cause the production of micelles. It can also dissolve protein. Tris may be used as a buffer. The presence of an ionic species such as magnesium chloride is typical of a phosphate-buffered system.

The process by which adenoviral vectors are produced, purified and stored can provide numerous opportunities for viral protein modifications that can possibly lead to adenovirus aggregation and decreased biological activity. For purified adenoviruses, aggregation has been reported to be a function of concentration, temperature, pH and storage container. Traditional analytical methods are not capable of distinguishing native monomeric viruses from aggregated particle populations.

### SUMMARY OF THE INVENTION

The present invention is based on the discovery that, in the context of adenovirus expressing thymidine kinase, and in particular as described in be prevented.

According to one aspect of the invention, a composition comprises a virus, a polyol and a zwitteronic compound. The virus is preferably an adenovirus. Typically, the adenovirus expresses thymidine kinase. Preferably, the polyol is glycerol. The zwitteronic compound is preferably HEPES.

A composition of the invention can be non-ionic and/or salt-free. It can also be serum-free.

According to another aspect of the invention, an assay for viral aggregation, comprises determining the size of the viral particles, e.g. wherein the particles are in admixture with a polyol or in a composition of the invention. The assay is preferably conducted by dynamic light scattering (DLS), also known as Photon Correlation Spectroscopy (PCS), and permits the analysis of viral particles in their native form during production and purification processes and thus enables the monitoring of possible particle aggregate formation.

### DESCRIPTION OF PREFERRED ENHANCEMENTS

In general terms, a composition according to the invention may comprise components as or of the type described in the prior art to which reference is made above; the content of each of these publications is incorporated herein by reference.

The virus may be a wild-type, or recombinant virus. The present invention can be utilised with a wide range of viruses. These include adenovirus, pox viruses, herpes viruses and lentiviruses. It is preferably an adenoviral vector of the type that can express a heterologus gene product. The gene product may be suitable for use in therapy, e.g. following resection of a brain tumour.

The viral preparation may be obtained by density fractionation, e.g. using a salt gradient, as is well known to those of ordinary skill in the art. The virus may also be purified and formulated with chromatographic purification methods (e.g. anion exchange and size exclusion) combined with tangential flow filtration (TFF).

The polyol may be added at this stage, or earlier. Many polyols are known, but glycerol is preferred. The concentration of glycerol in the formulation is preferably at least 2%, more preferably at least 5%, e.g. up to 30% (by weight or volume).

The composition is preferably non-ionic. There may be no salt added.

A sugar such as sucrose may be used in the invention, but its presence is not essential. Sucrose is known as a cryoprotectant, but it is important to avoid local pH effects on freezing. This is important because a composition of the invention will typically be lyophilised, and stored at low temperature, e.g. at −70° C.

In addition to a polyol, another component of the novel formulation is a zwitterion. Many such compounds are known and are suitable for use in compositions intended for therapeutic use. HEPES, i.e. 4-(2-hydroxy ethyl)-1-piperazineethanesulfonic acid, is preferred, but other internal organic amine/acids may be suitable. The concentration of this component is typically at least 1 mM, e.g. up to 10 or 20 mM.

The following Examples illustrate the invention.

### Example 1

Viral samples prepared for the use described in WO00/28059 were analysed for the presence of aggregates, when using a buffer comprising 5 mM HEPES, 20% glycerol, pH 7.8.

Samples were frozen after particle size analysis and re-analysed after one freeze-thaw. In the case of purified product formulated in the 20% glycerol-containing final formulation buffer there were no changes detected in the particle size profile of the sample. However, when the samples containing aggregated viruses were re-evaluated after one freeze and a thaw a clear change in particle size profile was detected. Similar results were obtained for all other tested samples.

Effect of repeated freeze/thaw on the particle size profile of a purified batch was evaluated. No aggregates could be detected even after five consecutive freeze and thaw cycles of the purified samples.

### Example 2

In this Example, particle size distributions were obtained using Nicomp 380 ZLS Particle Sizing System. This equipment uses DLS in the particle size analysis (http://www.pssnicomp.com/nicomptheory.htm): a laser beam is directed to the analysed sample and the intensity of scattered light is measuring from 90° angle. Scattering of light occurs when the laser beam collides with the solid particles present in the sample buffer. The intensity of scattered light in a particular direction changes periodically with time as the dispersed particles move around in the liquid. The smaller the particle size, the faster the particles move in the surrounding liquid and the faster the change in intensity of scattered light. Particle radius can be calculated based on data obtained from the fluctuations in the light intensity versus time profile. Currently, Zetasizer Nano S particle sizing equipment from Malvern Instruments is used for the determination of viral particle sizes. This equipment utilizes also DLS method for particle size analysis. According to the equipment manufacturers, DLS can size particles down to 1 nm, whereas methods based on laser diffraction can reliably size particles down to about 100 nm.

Batches of adenoviruses were made up, comprising (A) the product described in WO98/20027 and (B) the product described in WO00/28059, in final formulation buffer as described in Example 1. Batch (B) comprised samples before and after tangential flow filtration (TFF). This was done in order to study the effect of CsCl on the viral particle stability and aggregation in-process. Normally samples taken after TFF process contain negligible amounts of CsCl.

The batches were analysed for the presence of adenoviral aggregates, in order to evaluate the effect of glycerol on the adenovirus particle size measurements. Samples contained caesium chloride (CsCl). These samples were obtained after the adenovirus-containing bands collected from the ultracentrifuge tubes had been pooled and diluted 5-fold into 5 mM HEPES.

To obtain a positive aggregated sample, samples taken during the purification processes of were incubated at +37° C. for different times: 0 d, 2 d and 7 d without the addition of glycerol.

Glycerol was added into the selected samples to obtain final concentration of 20%. An equal volume of 5 mM HEPES was added into other set of HEPES pre-treated samples.

Samples containing viruses formulated into final formulation buffer (20% glycerol, 5 mM HEPES, pH 7.8) were incubated at +37° C. and analysed without any pre-treatments.

Viral aggregates were also prepared by means of immunoprecipitation using polyclonal adenovirus antibody (Abeam, Cambridge, UK; dilution 1:100). After particle size analysis, all samples were frozen and stored at −70° C. Samples that were incubated for 7 d at +37° C. and immunoprecipitated using polyclonal antibody were thawed once to determine the effect of formulation buffer on the sample particle size distribution.

It should be borne in mind that sample buffer composition can affect the obtained viral particle size. When the sample buffer contains 20% glycerol, the measured viral particle size is about 200 nm whereas in the sample that has been taken prior to TFF (without glycerol) the size is about 110 nm. According to the literature, the diameter of one intact adenovirus particle is about 80-90 nm. The differences between observed and reported virus particle sizes, especially in the case of glycerol-containing samples, can be explained by the effect of sample buffer composition. As glycerol makes the sample buffer more viscous, particle movement in the surrounding buffer is slowed down, leading to incorrect particle sizing. The Nicomp particle sizing equipment may be set on the assumption that the analyzed sample is formulated in an aqueous buffer with certain pre-set properties. When these pre-set measuring parameters are changed to correspond those of glycerol-containing buffer, the obtained results can be recalculated to obtain correct particle size readings.

It was found that glycerol seems to have an effect on the observed particle size but also appears to protect adenoviruses from aggregation. Aggregated viruses were not detected in any of the analysed samples that were incubated at +37° C. for 0 d and 2 d. After 7 d incubation at +37° C., aggregated particles appeared in the samples that were pre-treated just before particle size analysis. However, at 7 d time point the virus sample formulated in final formulation buffer appeared to be still free of detectable particle aggregates.

Also there was no detectable aggregation of viruses when glycerol was included in the sample buffer during the incubation at +37° C. Samples were frozen after each analysed time point. When frozen samples from 7 d time point were thawed and re-analysed, particle sizes of the pre-treated samples taken before TFF appeared to be affected whereas viruses in the final formulation buffer seemed to be intact.

Addition of polyclonal adenovirus antibody caused aggregation of viral particles. The presence of adenoviral aggregates in the antibody-treated sample was confirmed by transmission electron microscopy (TEM). There were no aggregates in the untreated control sample. However, big clusters of aggregated adenoviruses were detected in the antibody-treated sample.

There were no aggregates present in any of the tested purified samples formulated in the final formulation buffer. During the preliminary experiments, the limit of detection of Nicomp 380 ZLS Particle Sizing System was found to be 1×10¹¹ non-aggregated viral particles per ml. Currently Zetasizer Nano S particle sizing equipment is being used for the particle size analyses. Limit of detection for this equipment has been confirmed also to be 1×10¹¹ viral particles per ml. Aggregation of viral particles leads to decrease in the signal intensity.

1. A composition comprising a virus, a polyol and a zwitteronic compound.

2. The composition according to claim 1, wherein the virus is an adenovirus.

3. The composition according to claim 2, wherein the adenovirus expresses thymidine kinase.

4. The composition according to claim 1, wherein the polyol is glycerol.

5. The composition according to claim 1, wherein the zwitteronic compound is HEPES.

6. The composition according to claim 1, which is non-ionic.

7. The composition according to claim 1, which is lyophilised.

8. An assay for viral aggregation, which comprises analysing the size of the viral particles in a sample, wherein the particles are in admixture with a polyol, and determining from the size whether the sample contains substantially only acceptable, non-aggregated particles.

9. The assay according to claim 8, wherein the particles are in a composition comprising a virus, a polyol and a zwitteronic compound.

10. The assay according to claim 8, wherein the analysing is by dynamic light scattering.

11. The assay according to claim 8, wherein the size is no more than 200 nm.

12. The assay according to claim 11, wherein the size is no more than 100 nm.
